(19) United States
(12) Patent Application Publication
(10) Pub. No.: US 2026/0095730 A1
(43) Pub. Date: Apr. 2, 2026

(54) ENVIRONMENTAL MONITORING DEVICE, ENVIRONMENTAL MONITORING SYSTEM, AND ENVIRONMENTAL MONITORING METHOD

(71) Applicant: WNC Corporation, HSINCHU (TW)

(72) Inventors: CHIA-HSIN WU, HSINCHU (TW); CHIH-HSIANG PENG, HSINCHU (TW); CHU-CHI SUN, HSINCHU (TW); KO-WEN TSO, HSINCHU (TW); FU-MING KANG, HSINCHU (TW); TING-HUEI CHEN, HSINCHU (TW); SIN SZE IP, HSINCHU (TW)

(21) Appl. No.: 19/333,406

(22) Filed: Sep. 19, 2025

(30) Foreign Application Priority Data
Oct. 1, 2024 (TW) 113137481

(51) Int. Cl. H04W 4/38; G01N 33/00

(52) U.S. Cl. CPC H04W 4/38; G01N 33/0037; G01N 33/0063

(57) ABSTRACT

An environmental monitoring device, an environmental monitoring system and an environmental monitoring method. The environmental monitoring device includes a radio frequency identification (RFID) tag, a radio frequency (RF) reading module, and a determination circuit. The radio frequency identification tag is configured to transmit air quality information about an interior of a target device. The RF reading module is configured to receive and read the air quality information. The determination circuit is configured to determine whether the air quality information meets an air quality standard. When the air quality information does not meet the air quality standard, a warning message is generated.

### CROSS-REFERENCE TO RELATED PATENT APPLICATION

This application claims the benefit of priority to Taiwan Patent Application No. 113137481, filed on Oct. 1, 2024. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a monitoring equipment and a monitoring method, and more particularly, to an environmental monitoring device, an environmental monitoring system, and an environmental monitoring method.

### BACKGROUND OF THE DISCLOSURE

When the relative humidity inside a wafer transfer box is too high, it can cause oxidation, corrosion, short circuit, or current leakage in the semiconductor wafers inside the wafer transfer box (e.g., a front opening unified pod, FOUP), thereby reducing the performance and lifespan of the semiconductor wafers.

Currently, the conventional method used to control the relative humidity inside the wafer transfer box involves periodically injecting gas, such as clean dry air or nitrogen, into the interior of the wafer transfer box through an air conditioning system to reduce the relative humidity. However, the drawback of this conventional method is that even when the relative humidity inside the wafer transfer box is normal, the air conditioning system may still inject gas to reduce the relative humidity, leading to unnecessary waste of the gas. Alternatively, if the relative humidity is already too high before the gas is injected, defects may have already formed on the semiconductor wafers.

### SUMMARY OF THE DISCLOSURE

In response to the above-referenced technical inadequacies, the present disclosure provides an environmental monitoring device, an environmental monitoring system, and an environmental monitoring method for effectively improving on the issues associated with conventional methods.

In one aspect, the present disclosure provides an environmental monitoring device, which includes a radio frequency identification tag, a radio frequency reading module, and a determination circuit. The radio frequency identification tag is configured to transmit air quality information about an interior of a target device. The radio frequency reading module is configured to read the air quality information. The determination circuit is configured to determine whether the air quality information meets an air quality standard. When the air quality information does not meet the air quality standard, a warning message is generated.

In another aspect, the present disclosure provides an environmental monitoring system, which includes a target device, a radio frequency identification tag, a radio frequency reading module, and a determination circuit. The radio frequency identification tag is configured to transmit air quality information about an interior of the target device. The radio frequency reading module is configured to read the air quality information. The determination circuit is configured to determine whether the air quality information meets an air quality standard. When the air quality information does not meet the air quality standard, a warning message is generated.

In yet another aspect, the present disclosure provides an environmental monitoring method, including: transmitting, by a radio frequency identification tag, air quality information about an interior of a target device; reading, by a radio frequency reading module, the air quality information; determining, by a determination circuit, whether the air quality information meets an air quality standard; and generating a warning message when the air quality information does not meet the air quality standard.

One beneficial effect of the present disclosure is that through the environmental monitoring device, the environmental monitoring system, and the environmental monitoring method provided by the present disclosure, the relative humidity inside the target device can be monitored in real-time and accurately identified at the precise time point when the relative humidity is too high. This allows for the timely injection of gas to reduce the relative humidity inside the target device, thereby avoiding unnecessary waste of gas and reducing the possibility of defect formation due to high relative humidity.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of an environmental monitoring system according to a first embodiment of the present disclosure;

FIG. 2 is a flowchart of an environmental monitoring method of the environmental monitoring system shown in FIG. 1;

FIG. 3 is a schematic view of the environmental monitoring system according to a second embodiment of the present disclosure;

FIG. 4 is a flowchart of the environmental monitoring method of the environmental monitoring system shown in FIG. 3;

FIG. 5 is a schematic view of the environmental monitoring system according to a third embodiment of the present disclosure; and

FIG. 6 is a flowchart of the environmental monitoring method of the environmental monitoring system shown in FIG. 5.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

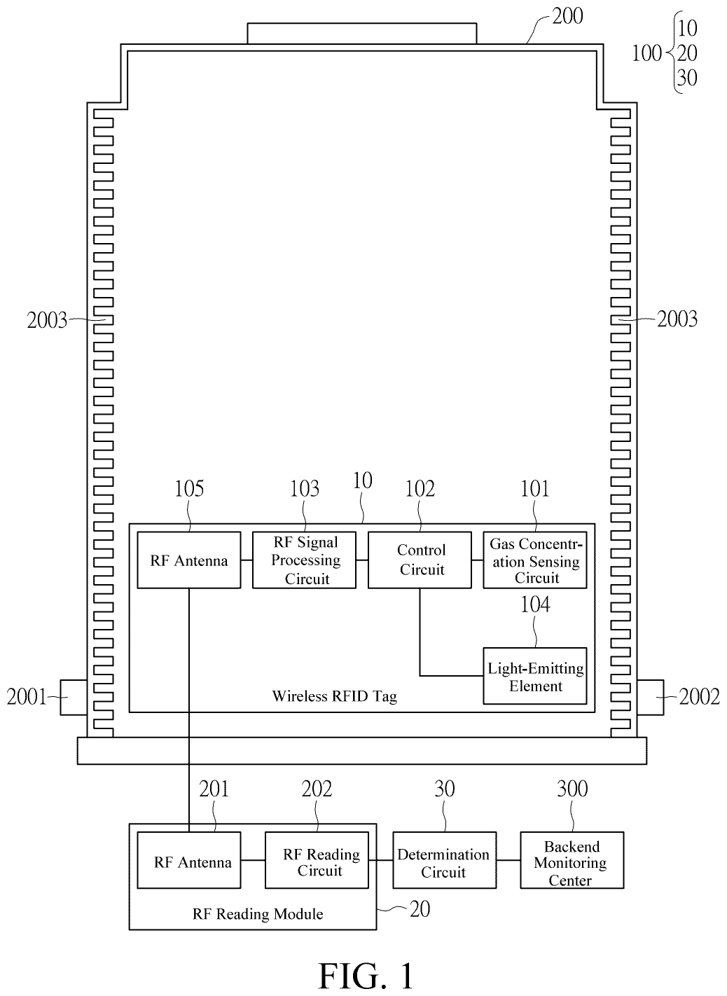

FIG. 1 is a schematic view of an environmental monitoring system according to a first embodiment of the present disclosure. Referring to FIG. 1, the environmental monitoring system includes an environmental monitoring device 100, a target device 200, and a backend monitoring center 300. In the present embodiment, the target device 200 is a wafer transfer box or a front opening unified pod (FOUP), but the present disclosure is not limited thereto. In other embodiments, the target device 200 may be a server cabinet or a battery box.

The wafer transfer box includes an inlet 2001, an outlet 2002, and a plurality of wafer slots 2003 at different height positions. The inlet 2001 is used to inject clean dry air or nitrogen, and each of the wafer slots 2003 is configured to hold a wafer.

The environmental monitoring device 100 includes a radio frequency identification (RFID) tag 10, a radio frequency (RF) reading module 20, and a determination circuit 30. The radio frequency identification tag 10 may be a passive radio frequency identification tag, and the determination circuit 30 may be an embedded controller, an application-specific integrated circuit (ASIC), a field-programmable gate array (FPGA), a microcontroller unit (MCU), a system on a chip (SOC), or any combination thereof.

The radio frequency identification tag 10 is positioned below a lowest one of the wafer slots 2003. The radio frequency reading module 20 and the determination circuit 30 are both positioned outside the target device 200. The radio frequency reading module 20 includes a radio frequency antenna 201 and a radio frequency reading circuit 202, with the radio frequency antenna 201 electrically connected to the radio frequency reading circuit 202 through a radio frequency cable.

When the radio frequency antenna 201 receives an external radio frequency signal, the radio frequency antenna 201 transmits the radio frequency signal to the radio frequency reading circuit 202, which reads data in the radio frequency signal. The radio frequency reading circuit 202 can also generate a radio frequency signal embedded with data and transmit the radio frequency signal through the radio frequency antenna 201.

The radio frequency reading circuit 202 is electrically connected to the determination circuit 30 through an Ethernet cable, and the determination circuit 30 is connected to the backend monitoring center 300 through a network connection.

The radio frequency identification tag 10 is configured to detect an air quality information inside (i.e., from an interior of) the wafer transfer box, and the determination circuit 30 determines whether the air quality information meets an air quality standard.

The air quality information includes oxygen concentration. The radio frequency identification tag 10 includes a gas concentration sensing circuit 101, a control circuit 102, a radio frequency signal processing circuit 103, a light-emitting element 104, and a radio frequency antenna 105. The control circuit 102 is electrically connected to the gas concentration sensing circuit 101, the radio frequency signal processing circuit 103, and the light-emitting element 104. The radio frequency signal processing circuit 103 is electrically connected to the radio frequency antenna 105. The control circuit 102 may be an embedded controller, an application-specific integrated circuit (ASIC), a field-programmable gate array (FPGA), a microcontroller unit (MCU), a system on a chip (SOC), or any combination thereof.

In other embodiments, the radio frequency identification tag 10 may be an active radio frequency identification tag with a built-in battery.

The gas concentration sensing circuit 101 is an oxygen concentration sensor that is configured to detect the oxygen concentration inside the wafer transfer box and that is configured to output the oxygen concentration to the control circuit 102. The control circuit 102 is configured to transmit the oxygen concentration to the radio frequency signal processing circuit 103.

The radio frequency signal processing circuit 103 is configured to generate a radio frequency signal embedded with the oxygen concentration and transmit the radio frequency signal through the radio frequency antenna 105. The radio frequency antenna 201 of the radio frequency reading module 20 is configured to receive the radio frequency signal from the radio frequency identification tag 10 and transmit the radio frequency signal to the radio frequency reading circuit 202.

The radio frequency reading circuit 202 is configured to read the oxygen concentration in the radio frequency signal and transmit the oxygen concentration to the determination circuit 30.

The determination circuit 30 is configured to calculate the nitrogen concentration inside (i.e., from the interior of) the wafer transfer box based on the oxygen concentration and determine whether the nitrogen concentration inside the wafer transfer box is less than a nitrogen concentration threshold (e.g., 94%).

When the determination circuit 30 determines that the nitrogen concentration inside the wafer transfer box is less than the nitrogen concentration threshold, the determination circuit 30 generates an alert signal and transmits the alert signal to the backend monitoring center 300. Simultaneously, the determination circuit 30 drives the light-emitting element 104 of the radio frequency identification tag 10 to emit light, with the alert signal and the light from the light-emitting element 104 serving as two different warning messages. In other words, as used herein, the term ‘warning message’ broadly refers to any alert information, and may include, but not limited to, a transmittable signal, a visual message, an audible or visual signal such as sound or light, or a combination thereof.

In other embodiments, the light-emitting element 104 of the radio frequency identification tag 10 can be replaced with a buzzer. When the determination circuit 30 determines that the nitrogen concentration is less than the nitrogen concentration threshold, the determination circuit 30 drives the buzzer of the radio frequency identification tag 10 to emit sound, with the sound serving as a warning message.

In this manner, whether it is the management personnel at the backend monitoring center 300 or the management personnel in the factory, they can know that the nitrogen concentration inside the wafer transfer box on the production line is currently too low by seeing the warning message. Subsequently, the management personnel can check whether there is a leakage situation occurring in the wafer transfer box.

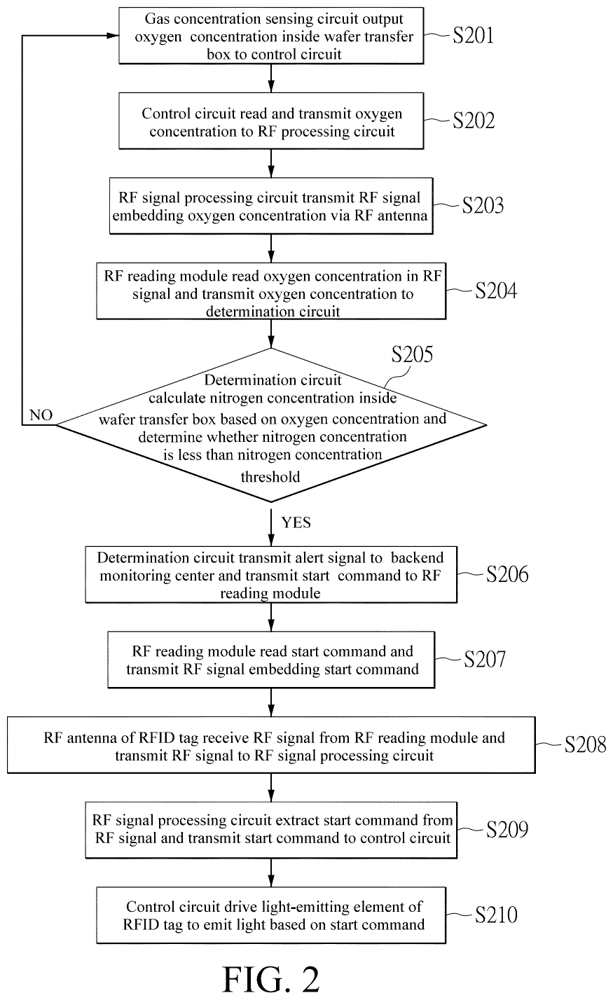

FIG. 2 is a flowchart of an environmental monitoring method of the environmental monitoring system shown in FIG. 1. Referring to FIGS. 1 and 2, in step S201, the gas concentration sensing circuit 101 outputs the oxygen concentration inside the wafer transfer box to the control circuit 102, where the gas concentration sensing circuit 101 is an oxygen concentration sensor.

In step S202, the control circuit 102 reads the oxygen concentration and transmits the oxygen concentration to the radio frequency signal processing circuit 103.

In step S203, the radio frequency signal processing circuit 103 transmits a radio frequency signal embedded with the oxygen concentration through the radio frequency antenna 105.

In step S204, the radio frequency reading module 20 reads the oxygen concentration in the radio frequency signal and transmits the oxygen concentration to the determination circuit 30.

In step S205, the determination circuit 30 calculates the nitrogen concentration inside the wafer transfer box based on the oxygen concentration and determines whether the nitrogen concentration is less than the nitrogen concentration threshold.

When the determination circuit 30 determines that the nitrogen concentration is less than the nitrogen concentration threshold, the process proceeds to the step S206.

When the determination circuit 30 determines that the nitrogen concentration is not less than the nitrogen concentration threshold, the process returns to the step S201.

In the step S206, the determination circuit 30 transmits an alert signal to the backend monitoring center 300 and transmits a start command to the radio frequency reading module 20, where the alert signal constitutes a warning message.

In step S207, the radio frequency reading module 20 reads the start command and transmits a radio frequency signal embedded with the start command.

In step S208, the radio frequency antenna 105 of the radio frequency identification tag 10 receives the radio frequency signal from the radio frequency reading module 20 and transmits the radio frequency signal to the radio frequency signal processing circuit 103.

In step S209, the radio frequency signal processing circuit 103 extracts the start command from the radio frequency signal and transmits the start command to the control circuit 102.

In step S210, the control circuit 102 drives the light-emitting element 104 of the radio frequency identification tag 10 to emit light based on the start command, with the light from the light-emitting element 104 serving as another warning message.

Specifically, the gas concentration sensing circuit 101 is used to detect the oxygen concentration inside the wafer transfer box, and the determination circuit 30 calculates the nitrogen concentration inside the wafer transfer box based on the oxygen concentration. When the determination circuit 30 determines that the nitrogen concentration inside the wafer transfer box is less than 94%, it indicates that there may be a possible leakage situation.

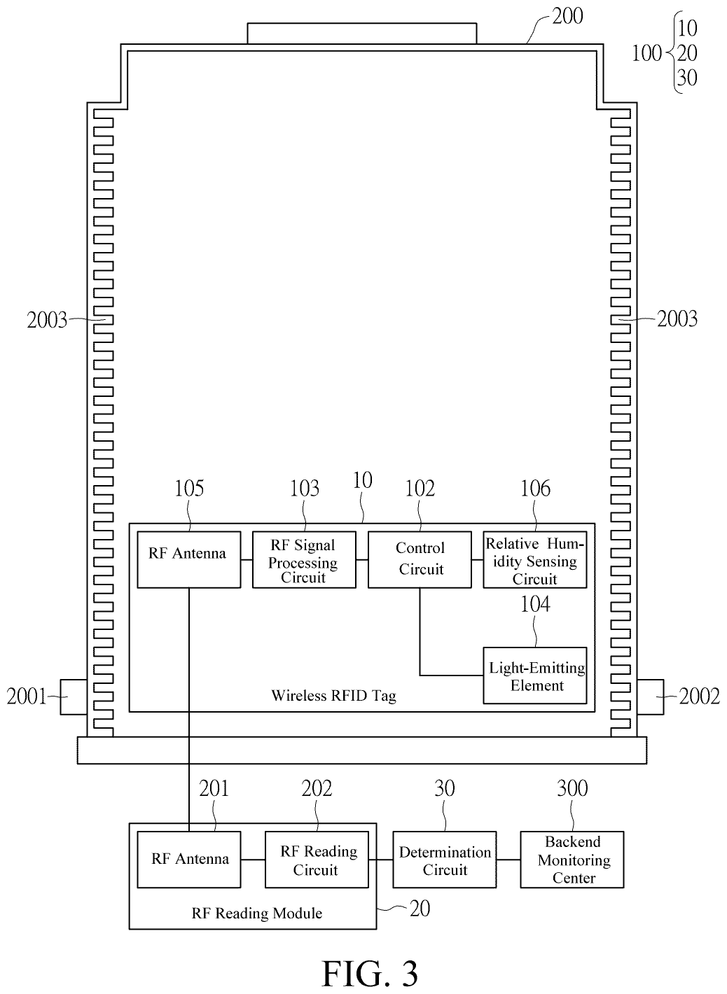

FIG. 3 is a schematic view of the environmental monitoring system according to a second embodiment of the present disclosure. Referring to FIG. 3, compared to FIG. 1, the difference is that the gas concentration sensing circuit 101 of the radio frequency identification tag 10 is replaced with a relative humidity sensing circuit 106, which is electrically connected to the control circuit 102.

The radio frequency identification tag 10 measures the relative humidity inside the wafer transfer box through the relative humidity sensing circuit 106, and the radio frequency signal processing circuit 103 of the radio frequency identification tag 10 is configured to generate a radio frequency signal embedding the relative humidity and to transmit the radio frequency signal through the radio frequency antenna 105.

The radio frequency reading module 20 is configured to read the relative humidity in the radio frequency signal and transmit the relative humidity to the determination circuit 30.

The determination circuit 30 is configured to determine whether the relative humidity is greater than a relative humidity threshold (e.g., 6%). When the determination circuit 30 determines that the relative humidity is greater than the relative humidity threshold, the determination circuit 30 transmits an alert signal to the backend monitoring center 300 and drives the light-emitting element 104 of the radio frequency identification tag 10 to emit light, with the alert signal and the light from the light-emitting element 104 serving as two different warning messages.

In this manner, whether it is the management personnel at the backend monitoring center 300 or the management personnel in the factory, they can know that the relative humidity inside the wafer transfer box on the production line is currently too high by seeing the warning message. Subsequently, the management personnel can inject clean dry air into the interior of the wafer transfer box to reduce the relative humidity, thereby avoiding oxidation, corrosion, or short circuit phenomena in the wafers due to high relative humidity.

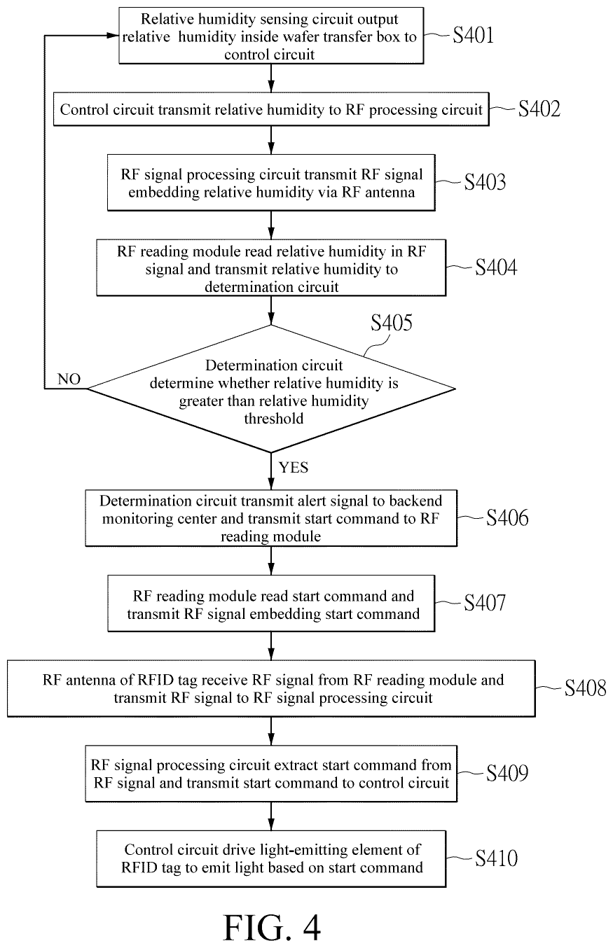

FIG. 4 is a flowchart of the environmental monitoring method of the environmental monitoring system shown in FIG. 3. The environmental monitoring method of FIG. 4 includes steps S401 to S410. Compared with FIG. 2, the differences are described as follows, and the similar steps (S406-S410) are not described herein.

In the step S401, the relative humidity sensing circuit 106 outputs the relative humidity inside the wafer transfer box to the control circuit 102.

In the step S402, the control circuit 102 transmits the relative humidity to the radio frequency signal processing circuit 103.

In the step S403, the radio frequency signal processing circuit 103 transmits a radio frequency signal embedded with the relative humidity through the radio frequency antenna 105.

In the step S404, the radio frequency reading module 20 reads the relative humidity in the radio frequency signal and transmits the relative humidity to the determination circuit 30.

In the step S405, the determination circuit 30 determines whether the relative humidity is greater than the relative humidity threshold.

When the determination circuit 30 determines that the relative humidity is greater than the relative humidity threshold, the process proceeds to the step S406.

When the determination circuit 30 determines that the relative humidity is not greater than the relative humidity threshold, the process returns to the step S401.

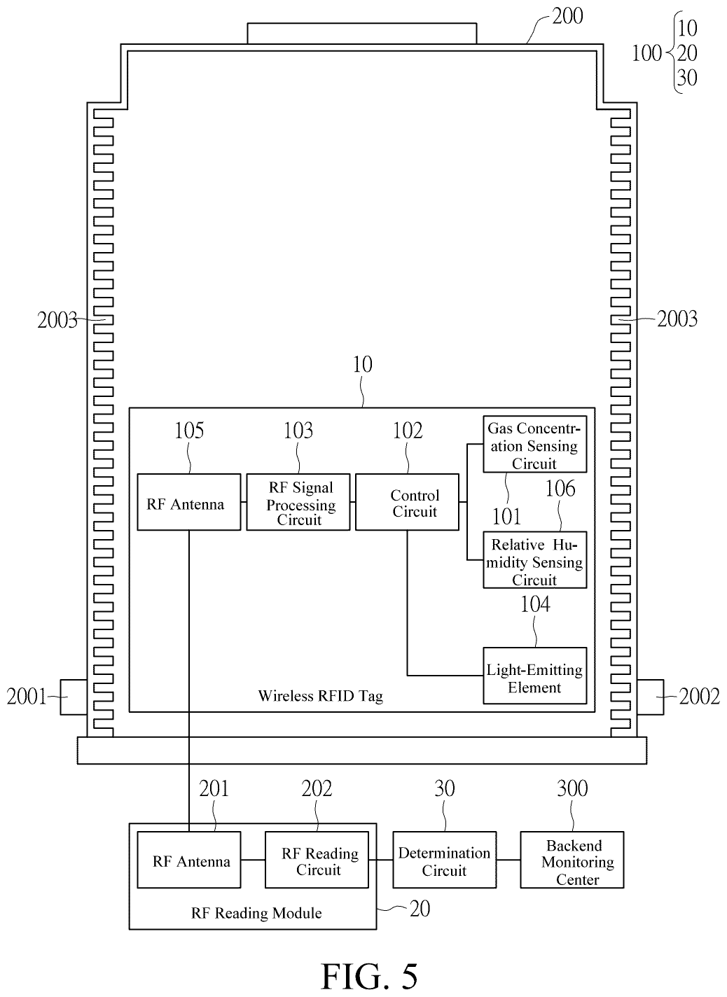

FIG. 5 is a schematic view of the environmental monitoring system according to a third embodiment of the present disclosure. The difference between FIG. 5 and FIGS. 1 and 3 is that the radio frequency identification tag 10 in FIG. 5 includes both the gas concentration sensing circuit 101 and the relative humidity sensing circuit 106, with the gas concentration sensing circuit 101 and the relative humidity sensing circuit 106 electrically connected to the control circuit 102.

The radio frequency signal processing circuit 103 of the radio frequency identification tag 10 is configured to generate a radio frequency signal embedded with the oxygen concentration and the relative humidity and transmit the radio frequency signal through the radio frequency antenna 105.

The radio frequency reading circuit 202 reads the oxygen concentration and the relative humidity in the radio frequency signal.

The determination circuit 30 calculates the nitrogen concentration inside the wafer transfer box based on the oxygen concentration and determines whether the nitrogen concentration is less than the nitrogen concentration threshold and whether the relative humidity is greater than the relative humidity threshold. When the nitrogen concentration is less than the nitrogen concentration threshold and/or the relative humidity is greater than the relative humidity threshold, the determination circuit 30 transmits an alert signal to the backend monitoring center 300 and drives the light-emitting element 104 of the radio frequency identification tag 10 to emit light.

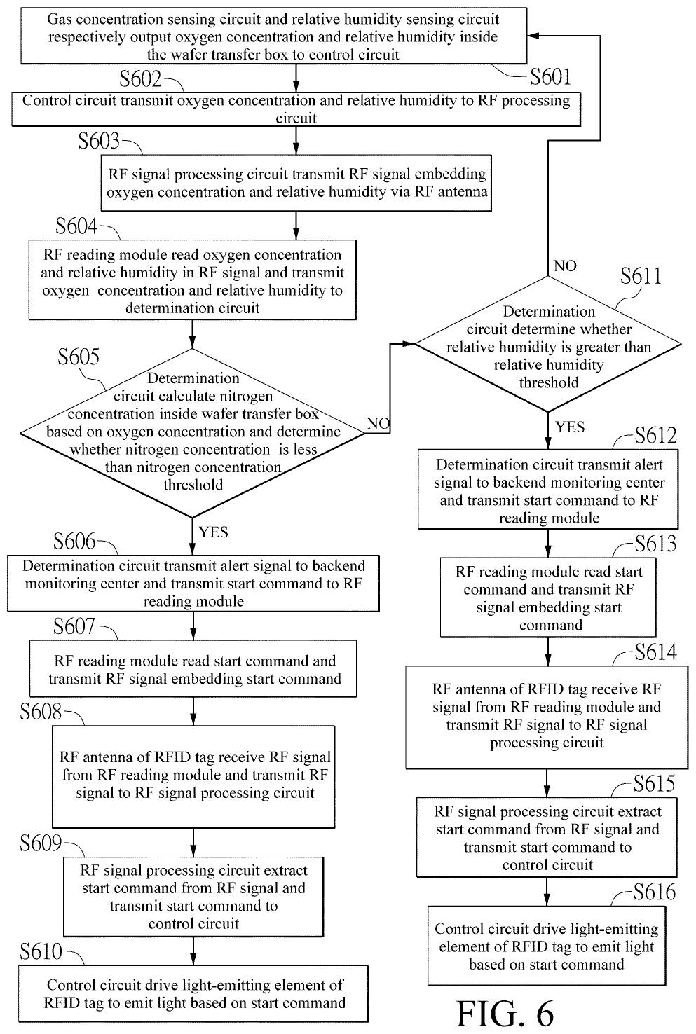

FIG. 6 is a flowchart of the environmental monitoring method of the environmental monitoring system shown in FIG. 5. The environmental monitoring method of FIG. 6 includes steps S601 to S616. Compared to FIG. 2 and FIG. 4, the differences are described as follows.

In the step S601, the gas concentration sensing circuit 101 and the relative humidity sensing circuit 106 respectively output the oxygen concentration and the relative humidity inside the wafer transfer box to the control circuit 102.

In the step S602, the control circuit 102 transmits the oxygen concentration and the relative humidity to the radio frequency signal processing circuit 103.

In the step S603, the radio frequency signal processing circuit 103 transmits a radio frequency signal embedded with the oxygen concentration and the relative humidity through the radio frequency antenna 105.

In the step S604, the radio frequency reading module 20 reads the oxygen concentration and the relative humidity in the radio frequency signal and transmits the oxygen concentration and the relative humidity to the determination circuit 30.

In the step S605, the determination circuit 30 calculates the nitrogen concentration inside the wafer transfer box based on the oxygen concentration and determines whether the nitrogen concentration is less than the nitrogen concentration threshold.

When the determination circuit 30 determines that the nitrogen concentration is less than the nitrogen concentration threshold, the process proceeds to the step S606.

When the determination circuit 30 determines that the nitrogen concentration is not less than the nitrogen concentration threshold, the process proceeds to the step S611.

In the step S611, the determination circuit 30 determines whether the relative humidity is greater than the relative humidity threshold.

When the determination circuit 30 determines that the relative humidity is greater than the relative humidity threshold, the process proceeds to the step S612. When the determination circuit 30 determines that the relative humidity is not greater than the relative humidity threshold, the process returns to the step S601.

In the step S612, the determination circuit 30 transmits an alert signal to the backend monitoring center 300 and transmits a start command to the radio frequency reading module 20.

In the step S613, the radio frequency reading module 20 reads the start command and transmits a radio frequency signal embedding the start command.

In the step S614, the radio frequency antenna 105 of the radio frequency identification tag 10 receives the radio frequency signal from the radio frequency reading module 20 and transmits the radio frequency signal to the radio frequency signal processing circuit 103.

In the step S615, the radio frequency signal processing circuit 103 extracts the start command from the radio frequency signal and transmits the start command to the control circuit 102.

In the step S616, the control circuit 102 drives the light-emitting element 104 of the radio frequency identification tag 10 to emit light based on the start command.

In other embodiments, the control circuit 102 can be omitted, and the determination circuit 30 can be disposed inside the radio frequency identification tag 10. The determination circuit 30 placed inside the target device 200 is configured to calculate the air quality information (e.g., nitrogen concentration and/or relative humidity) of the interior of the target device 200 and determine whether the air quality information meets the air quality standard. When the air quality information does not meet the air quality standard, the determination circuit 30 itself generates a warning message or drives other components to generate a warning message (e.g., sound or light). The determination circuit 30 transmits the determination result of the air quality to the radio frequency reading module 20 arranged outside the target device 200. The radio frequency reading module 20 is configured to read the determination result of the air quality and transmit the determination result to the backend monitoring center 300.

In other embodiments, the control circuit 102 of the radio frequency identification tag 10 that is disposed in the target device 200 is configured to calculate the air quality information about the interior of the target device 200. The radio frequency identification tag 10 transmits the air quality information through the radio frequency antenna 105 to the radio frequency reading module 20 arranged outside the target device 200. The radio frequency reading module 20 reads the air quality information and transmits the air quality information to the determination circuit 30 arranged outside the target device 200. The determination circuit 30 is configured to determine whether the air quality information meets the air quality standard. When the air quality information does not meet the air quality standard, the determination circuit 30 transmits an alert signal to the backend monitoring center 300 or drives other components to generate a warning message.

### BENEFITS OF THE EMBODIMENTS

One beneficial effect of the present disclosure is that through the environmental monitoring device, the environmental monitoring system, and the environmental monitoring method provided by the present disclosure, the relative humidity inside the target device can be monitored in real-time and accurately identified at the precise time point when the relative humidity is too high. This allows for the timely injection of gas to reduce the relative humidity inside the target device, thereby avoiding unnecessary waste of gas and reducing the possibility of defect formation due to high relative humidity.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An environmental monitoring device, comprising:
a radio frequency identification tag configured to transmit air quality information about an interior of a target device;
a radio frequency reading module configured to read the air quality information; and
a determination circuit configured to determine whether the air quality information meet an air quality standard;
wherein, when the air quality information does not meet the air quality standard, a warning message is generated.

2. The environmental monitoring device according to claim 1, wherein the air quality information comprises an oxygen concentration, the radio frequency identification tag comprises a gas concentration sensing circuit configured to output the oxygen concentration, and the determination circuit is configured to calculate a nitrogen concentration based on the oxygen concentration and is configured to determine whether the nitrogen concentration is less than a nitrogen concentration threshold; and wherein, when the nitrogen concentration is less than the nitrogen concentration threshold, the air quality information does not meet the air quality standard.

3. The environmental monitoring device according to claim 1, wherein the air quality information comprises a relative humidity, the radio frequency identification tag includes a relative humidity sensing circuit configured to output the relative humidity, and the determination circuit is configured to determine whether the relative humidity is greater than a relative humidity threshold; and wherein, when the relative humidity is greater than the relative humidity threshold, the air quality information does not meet the air quality standard.

4. The environmental monitoring device according to claim 1, wherein the air quality information comprises an oxygen concentration and a relative humidity, the radio frequency identification tag includes a gas concentration sensing circuit and a relative humidity sensing circuit, the gas concentration sensing circuit is configured to output the oxygen concentration, the relative humidity sensing circuit is configured to output the relative humidity, the determination circuit is configured to calculate a nitrogen concentration based on the oxygen concentration and is configured to determine whether the nitrogen concentration is less than a nitrogen concentration threshold, and the determination circuit is further configured to determine whether the relative humidity is greater than a relative humidity threshold; and wherein, when the nitrogen concentration is less than the nitrogen concentration threshold or the relative humidity is greater than the relative humidity threshold, the air quality information does not meet the air quality standard.

5. The environmental monitoring device according to claim 1, wherein the radio frequency identification tag further comprises a light-emitting element, and the warning message includes light emitted by the light-emitting element.

6. The environmental monitoring device according to claim 1, wherein the determination circuit is disposed in the radio frequency identification tag, and the radio frequency reading module is configured to read a determination result of the determination circuit and is configured to transmit the determination result to a backend monitoring center.

7. An environmental monitoring system, comprising:
a target device;
a radio frequency identification tag disposed inside the target device and configured to transmit air quality information about an interior of the target device;
a radio frequency reading module configured to read the air quality information; and
a determination circuit configured to determine whether the air quality information meets an air quality standard;
wherein, when the air quality information does not meet the air quality standard, a warning message is generated.

8. The environmental monitoring system according to claim 7, wherein the air quality information comprises an oxygen concentration, the radio frequency identification tag comprises a gas concentration sensing circuit configured to output the oxygen concentration, and the determination circuit is configured to calculate a nitrogen concentration based on the oxygen concentration and is configured to determine whether the nitrogen concentration is less than a nitrogen concentration threshold; and wherein, when the nitrogen concentration is less than the nitrogen concentration threshold, the air quality information does not meet the air quality standard.

9. The environmental monitoring system according to claim 7, wherein the air quality information comprises a relative humidity, the radio frequency identification tag includes a relative humidity sensing circuit configured to output the relative humidity, and the determination circuit is configured to determine whether the relative humidity is greater than a relative humidity threshold; and wherein, when the relative humidity is greater than the relative humidity threshold, the air quality information does not meet the air quality standard.

10. The environmental monitoring system according to claim 7, wherein the air quality information comprises an oxygen concentration and a relative humidity, the radio frequency identification tag comprises a gas concentration sensing circuit and a relative humidity sensing circuit, the gas concentration sensing circuit is configured to output the oxygen concentration, the relative humidity sensing circuit is configured to output the relative humidity, the determination circuit is configured to calculate a nitrogen concentration based on the oxygen concentration and is configured to determine whether the nitrogen concentration is less than a nitrogen concentration threshold, and the determination circuit is further configured to determine whether the relative humidity is greater than a relative humidity threshold; and wherein, when the nitrogen concentration is less than the nitrogen concentration threshold or the relative humidity is greater than the relative humidity threshold, the air quality information does not meet the air quality standard.

11. The environmental monitoring system according to claim 7, wherein the radio frequency identification tag further comprises a light-emitting element, and the warning message includes light emitted by the light-emitting element.

12. The environmental monitoring system according to claim 7, wherein the target device is a wafer transfer box.

13. The environmental monitoring system according to claim 7, wherein the determination circuit is disposed in the radio frequency identification tag, and the radio frequency reading module is configured to read a determination result of the determination circuit and is configured to transmit the determination result to a backend monitoring center.

14. An environmental monitoring method comprising:
transmitting, by a radio frequency identification tag, air quality information about an interior of a target device;
reading, by a radio frequency reading module, the air quality information;
determining, by a determination circuit, whether the air quality information meets an air quality standard; and
generating a warning message when the air quality information does not meet the air quality standard.

15. The environmental monitoring method according to claim 14, wherein the air quality information comprises an oxygen concentration, and the determination circuit calculates a nitrogen concentration based on the oxygen concentration; and wherein, when the determination circuit determines that the nitrogen concentration is less than a nitrogen concentration threshold, the air quality information does not meet the air quality standard.

16. The environmental monitoring method according to claim 14, wherein the air quality information comprises a relative humidity; and wherein, when the determination circuit determines that the relative humidity is greater than a relative humidity threshold, the air quality information does not meet the air quality standard.

17. The environmental monitoring method according to claim 14, wherein the air quality information comprises an oxygen concentration and a relative humidity, and the determination circuit calculates a nitrogen concentration based on the oxygen concentration; and wherein, when the determination circuit determines that the nitrogen concentration is less than a nitrogen concentration threshold and/or the relative humidity is greater than a relative humidity threshold, the air quality information does not meet the air quality standard.

18. The environmental monitoring method according to claim 14, wherein the radio frequency identification tag further comprises a light-emitting element, and the warning message includes light emitted by the light-emitting element.
